(19) United States
(12) Patent Application Publication
(10) Pub. No.: US 2004/0059180 A1
(43) Pub. Date: Mar. 25, 2004

(54) Basal mounting cushion frame component to facilitate extrinsic heart wall actuation

(75) Inventor: David Boyd Melvin, Loveland, OH (US)

Correspondence Address: WOOD, HERRON & EVANS, LLP, 2700 CAREW TOWER, 441 VINE STREET, CINCINNATI, OH, 45202, US

(73) Assignee: The University of Cincinnati

(21) Appl. No.: 10/667,877

(22) Filed: Sep. 22, 2003

Related U.S. Application Data
(60) Provisional application No. 60/413,004, filed on Sep. 23, 2002.

(51) Int. Cl. A61N 1/362

(52) U.S. Cl. 600/16; 600/37; 623/3.1

(57) ABSTRACT

An actuation system for assisting the operation of a natural heart that includes a dome structure configured for being coupled with a ventricular portion of the heart. The dome structure has at least one opening formed therein. The dome structure, proximate the opening, is configured to interface with at least one of an atrial chamber and a great vessel of the heart. A stabilizing element or an actuating element may be anchored to the dome structure for engaging a portion of the heart.

### RELATED APPLICATIONS

The application claims the benefit of the priority of U.S. Provisional Application Serial No. 60/413,004, filed Sep. 23, 2002, which application is incorporated herein in its entirety.

### FIELD OF THE INVENTION

This invention relates generally to assisting the natural heart in operation and, more specifically, to components to assist in actuating a wall of the natural heart.

### BACKGROUND OF THE INVENTION

The natural human heart and accompanying circulatory system are critical components of the human body and systematically provide the needed nutrients and oxygen for the body. As such, the proper operation of the circulatory system, and particularly, the proper operation of the heart, are critical in the life, health, and well-being of a person. A physical ailment or condition which compromises the normal and healthy operation of the heart can therefore be particularly critical and may result in a condition which must be medically remedied.

More specifically, the natural heart, or rather the cardiac tissue of the heart, can degrade for various reasons to a point where the heart can no longer provide sufficient circulation of blood for maintaining the health of a patient at a desirable level. In fact, the heart may degrade to the point of failure and thereby may not even be able to sustain life. To address the problem of a failing natural heart, solutions are offered to provide ways in which circulation of blood might be maintained. Some solutions involve replacing the heart. Other solutions are directed to maintaining operation of the existing heart.

One such solution has been to replace the existing natural heart in a patient with an artificial heart or a ventricular assist device. In using artificial hearts and/or assist devices, a particular problem stems from the fact that the materials used for the interior lining of the chambers of an artificial heart are in direct contact with the circulating blood. Such contact may enhance undesirable clotting of the blood, may cause a build-up of calcium, or may otherwise inhibit the blood's normal function. As a result, thromboembolism and hemolysis may occur. Additionally, the lining of an artificial heart or a ventricular assist device can crack, which inhibits performance, even when the crack is at a microscopic level. Such drawbacks have limited use of artificial heart devices to applications having too brief of a time period to provide a real lasting health benefit to the patient.

An alternative procedure also involves replacement of the heart and includes a transplant of a heart from another human or animal into the patient. The transplant procedure requires removing an existing organ (i.e., the natural heart) from the patient for substitution with another organ (i.e., another natural heart) from another human, or potentially, from an animal. Before replacing an existing organ with another, the substitute organ must be matched to the recipient, which can be, at best, difficult, time consuming, and expensive to accomplish. Furthermore, even if the transplanted organ matches the recipient, a risk exists that the recipient's body will still reject the transplanted organ and attack it as a foreign object. Moreover, the number of potential donor hearts is far less than the number of patients in need of a natural heart transplant. Although use of animal hearts would lessen the problem of having fewer donors than recipients, there is an enhanced concern with respect to the rejection of the animal heart.

Rather than replacing the patient's heart, other solutions attempt to continue to use the existing heart and associated tissue of the patient. In one such solution, attempts have been made to wrap skeletal muscle tissue around the natural heart to use as an auxiliary contraction mechanism so that the heart may pump. As currently used, skeletal muscle cannot alone typically provide sufficient and sustained pumping power for maintaining proper and desirable circulation of blood through the circulatory system of the body. This is especially true for those patients with severe heart failure. Another system developed for use with an existing heart for sustaining the circulatory function and pumping action of the heart, is an external bypass system, such as a cardiopulmonary (heart-lung) machine. Typically, bypass systems of this type are complex and large, and, as such, are limited to short term use, such as in an operating room during surgery, or when maintaining the circulation of a patient while awaiting receipt of a transplant heart. The size and complexity effectively prohibit use of bypass systems as a long term solution, as they are rarely portable devices. Furthermore, long term use of a heart-lung machine can damage the blood cells and blood borne products, resulting in post surgical complications such as bleeding, thromboembolism, and increased risk of infection.

Still another solution for maintaining the existing natural heart as the pumping device involves enveloping a substantial portion of the natural heart, such as the entire left and right ventricles, with a pumping device for rhythmic compression. That is, the exterior wall surfaces of the heart are contacted and the heart walls are compressed to change the volume of the heart and thereby pump blood out of the chambers. Although somewhat effective as a short term treatment, the pumping device has not been suitable for long term use. Typically, with such compression devices, heart walls are concentrically compressed. The compressive movement patterns, which reduce a chamber's volume and distort the heart walls, may not necessarily facilitate valve closure (which can lead to valve leakage).

Therefore, mechanical pumping of a patient's existing heart, such as through mechanical compression of the ventricles or some other action thereon, must address these issues and concerns in order to establish the efficacy of long term mechanical or mechanically assisted pumping. Specifically, the ventricles must rapidly and passively refill at low physiologic pressures, and the valve functions must be physiologically adequate. The myocardial blood flow of the heart also must not be impaired by the mechanical device. Still further, the left and right ventricle pressure independence must be maintained within the heart.

Mechanical ventricular wall actuation of the weakened existing heart of a patient has shown promise. As such, devices have been invented for mechanically assisting the pumping function of the heart, and specifically for externally actuating a heart wall, such as a ventricular wall, to assist in such pumping functions.

Specifically, U.S. Pat. No. 5,957,977, which is incorporated herein by reference in its entirety, discloses an actuation system for the natural heart utilizing internal and external support structures. That patent provides an internal and external framework mounted internally and externally with respect to the natural heart, and an actuator device or activator mounted to the framework for providing cyclical forces to deform one or more walls of the heart, such as the left ventricular wall. The invention of U.S. patent application Ser. No. 09/850,554, which has issued as U.S. Pat. No. 6,592,619, further adds to the art of U.S. Pat. No. 5,957,977 and that patent is also incorporated herein by reference in its entirety. The application specifically sets forth various embodiments of activator or actuator devices which are suitable for deforming the heart walls and supplementing and/or providing the pumping function for the natural heart.

While the actuation systems of those patents provide a desirable actuation of the natural heart, it is further desirable to improve upon the interface between the actuation system and the heart. Specifically, the coupling between the internal and external framework elements of the actuation system occurs across tissue. For example, transmural cords extend between semi-rigid internal valve annular rings and an external transverse arc of a yoke coupled to the outside of the heart. Due to overtightening of the cords when they are positioned into place, and/or to swelling of the tissue afterward, there may be compression of myocardial tissue and traversing of coronary artery branches. Therefore, it is desirable to avoid such tissue compression and other such issues associated with coupling the internal and external framework elements of an actuation system.

It is further desirable to achieve such goals while still providing sufficient anchoring for the various components of the actuation system. It is still further desirable to provide a counter-force to stabilize the base of the ventricular mass during application of deforming forces to the free walls and/or septum of the ventricle or ventricles.

Still further, it is an objective to provide desirable actuation of the heart to achieve a long-term solution to heart weakening or heart failure. These objectives and other objectives and advantages of the present invention will be set forth and will become more apparent in the description of the embodiments below.

### BRIEF DESCRIPTION OF THE DRAWINGS

The accompanying drawings, which are incorporated in and constitute a part of this specification, illustrate embodiments of the invention and, together with a general description of the invention given below, serve to explain the principles of the invention.

FIG. 1a is a perspective view of an excised heart with an embodiment of a basal ‘dome’ or ‘cap’ in place seen from right ventricular aspect.

FIG. 1b is a photographic view of an embodiment of a dome.

FIG. 1c is a photographic view of an embodiment of the invention on a heart structure.

FIG. 1d is a photographic view of an embodiment showing the yoke arc in position with the dome.

FIG. 2a is a top view of an embodiment of the invention.

FIG. 2b is a perspective view of an embodiment of the invention.

FIG. 2c is a sectional view through the plane defined by A-A′ in 2a.

FIG. 3a is a perspective view of an embodiment of a dome with flared extensions to support the external walls of great vessels and/or atria.

FIG. 3b is a sectional view of a single opening of a dome in which the flared extension is a separate part from the dome.

FIG. 4a is a sectional view illustrating one means of securing margins of a dome opening to walls of the great vessels or atria.

FIG. 4b is a sectional view of another securing means including an interlocking ring, both on the left atrium as an example.

FIG. 5a is a sectional view of means of securing a dome directly or indirectly to the valve annuli, such as by anchoring the dome anchored by pledgeted sutures fixed to two different parts of the aortic valve annulus.

FIG. 5b is a sectional view of a suture anchored to the mitral annulus by means of an anuloplasty ring and extending through atrial wall to the plane of the dome opening.

FIG. 5c is a sectional view of a special widely flared anuloplasty ring whose upper margin reaches the plane of the dome opening, to which it is fixed by at least one transmural suture.

FIG. 6 is a sectional view through the aortic root, showing part of a basal dome and a cushion between the dome and the cardiac structures on which it rests.

FIG. 7 is a sectional view of a basal dome constructed for placement on a heart that remains in situ—i.e., has not been excised, with the intent of not performing an autotransplant.

FIGS. 8a and 8b are partial perspective views of examples of means of connecting other components of the actuating system to the basal dome.

### DETAILED DESCRIPTION OF EMBODIMENTS OF THE INVENTION

For the purposes of illustrating the invention, the following parts list corresponds to the Figures listed above and included herewith.

Part Numbers

1. dome

2. left ventricle

3. right ventricle

4. left atrium

5. right atrium

6. aorta

7. pulmonary artery

8. opening in the dome for the left atrium

9. opening in the dome for the right atrium

10. opening in the dome for the aorta

11. opening in the dome for the pulmonary artery

12. flared extensions along the external walls of atria or great vessels, made intrinsic to the dome

13. a flared extension along the external wall of the aorta, made as a separate, insertable part

14. sutures fastening inner margin of dome opening to atrium

15. mitral valve

16. -

17. right ventricle

18. interlocking ring used to fasten atrial wall to inner surface of dome opening

19. proximal right coronary artery

20. right coronary cusp of the aortic valve, shown in section through its center

21. free margin of the noncoronary cusp of the aortic valve

22. aortic wall, shown in longitudinal section

23. commissure between the left coronary cusp and the noncoronary cusp of the aortic valve

24. noncoronary cusp of the aortic valve

25. cushion

26. left coronary cusp of the aortic valve

27. proximal left coronary artery

28. septal leaflet of the tricuspid valve

29. another part of the heart-actuating system—generally either additional stabilizing component or an actuating component—that is being attached to the basal dome

30. a suture

31. conventional type of mitral anuloplasty ring

32. extended, flared type of mitral anuloplasty ring

33. Teflon® felt or other type of anchoring pledget stabilizing a suture below the commisure between the right coronary cusp and noncoronary cusp of the aortic valve annulus

34. Teflon® felt or other type of anchoring pledget stabilizing a suture above the commisure between the left coronary cusp and noncoronary cusp of the aortic valve annulus

35. Ring structure

36. Flex point of ring structure

37. Separation point of ring structure

38. Clip attachment structure

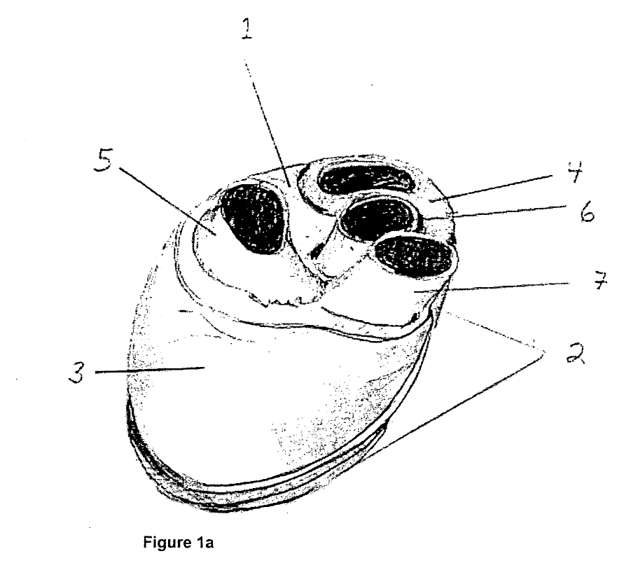

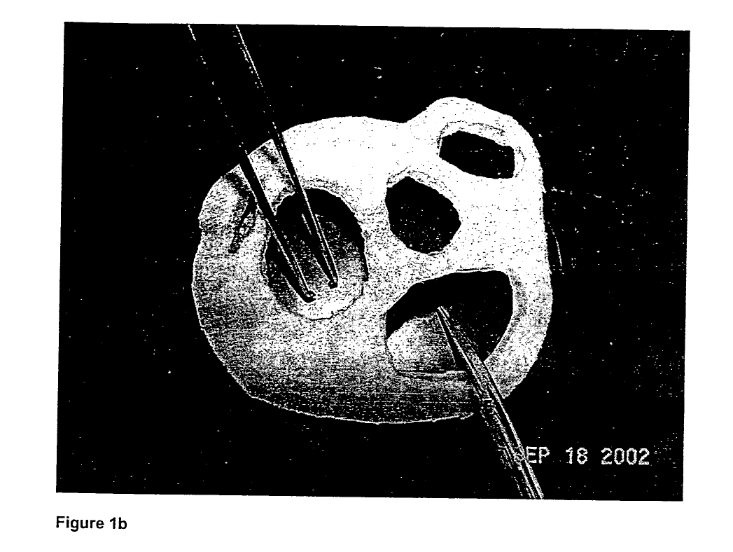

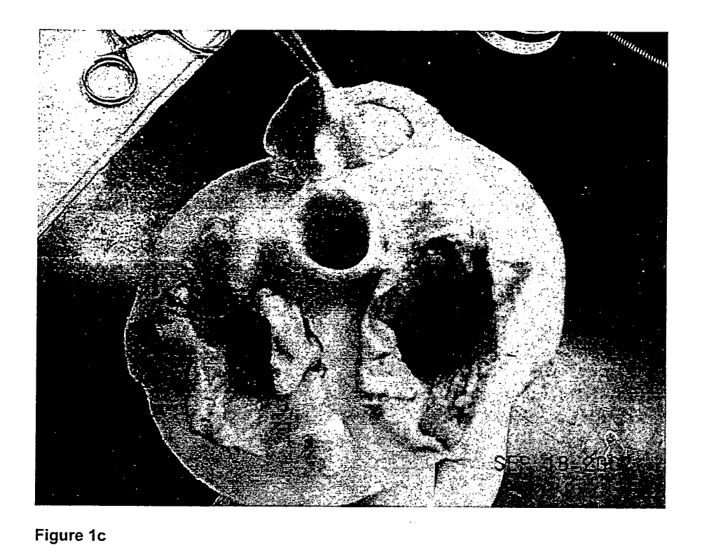

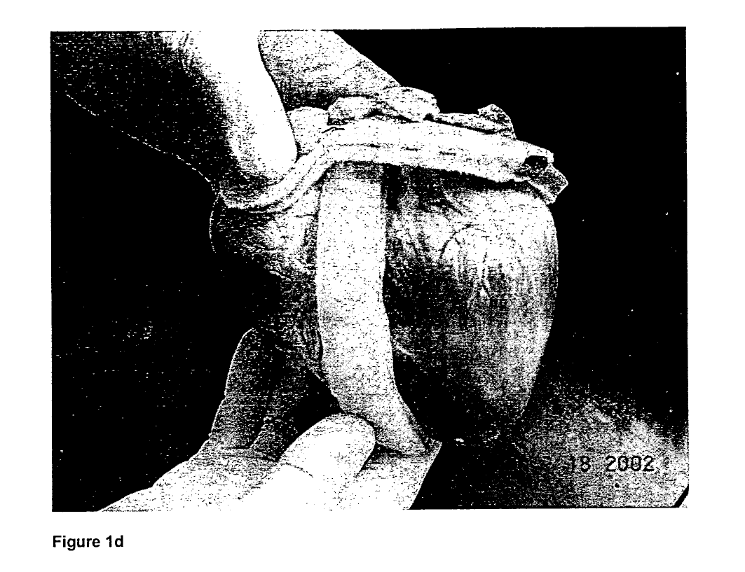

In one embodiment of the present invention, a dome-shaped cushion or dome is configured to rest on the base of one or both cardiac ventricles, providing a stable anchoring point for other internal and external components or systems that stabilize and/or mechanically actuate the ventricular walls for supplementation or replacement of myocardial contraction in the pumping of blood. FIGS. 1a to 1d illustrate one such dome-shaped cushion for use on a heart and with a support or actuation system. The invention, in another aspect, also reduces/eliminates compression of myocardial tissue and traversing coronary artery branches, as well as providing other benefits.

The dome is placed over the atria and has appropriated openings for various vascular components in that heart area. To facilitate placement of the dome, the separation of the two atria is desired in one aspect, and such separation generally need to extend closer to the atrioventricular valves than is encountered in natural anatomy. Therefore, the heart is prepared for placement of the dome 1. This may be done by first “developing” the intra-atrial groove by separating the cleavage line of fat in a manner commonly employed in mitral valve access, and well known to those familiar with the art of cardiac surgery, then incising one of the atria (preferably the right) at the margin of the septum, and finally closing the resulting defect with either a patch (e.g., prosthetic or pericardial) or direct suture.

In one embodiment, the dome 1 may be very flexible. For example, it may be formed of a soft fabric, or of an elastic material, such as of an elastomer with or without fiber or fabric reinforcement. Alternatively, the dome 1 may be rigid, such as of a cast or machined thermoplastic. In another alternative embodiment, the dome has regions that range within and between each of these extremes of general flexural behavior.

Referring to FIGS. 2a-2c, the dome 1 conforms to the general shape of the basal surface of the left, right, or both ventricles 2,3, and is concave toward the ventricle(s). There are openings 8-11 for each of the chambers (atria 4,5 and great vessels) surrounding the respective valves. For example, four openings would exist for a biventricular dome and two openings are used for a univentricular dome. Peripheral to the margins of the openings, the dome margins conform to the general shape of the basal-most portions of the ventricles.

Referring to FIG. 2b, various openings 8-11 may be seen. Opening 8 is for the left atrium, while opening 9 is for the right atrium (see FIG. 1a). Opening 10 in the dome 1 is for accommodation of the aorta 6. Opening 11 similarly is for accommodating the pulmonary artery 7 (see FIG. 1a).

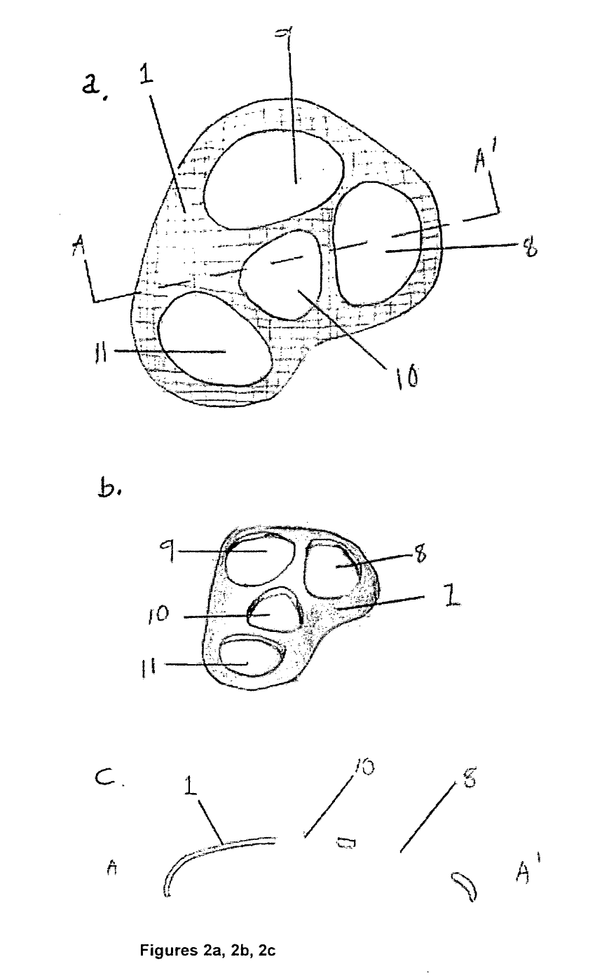

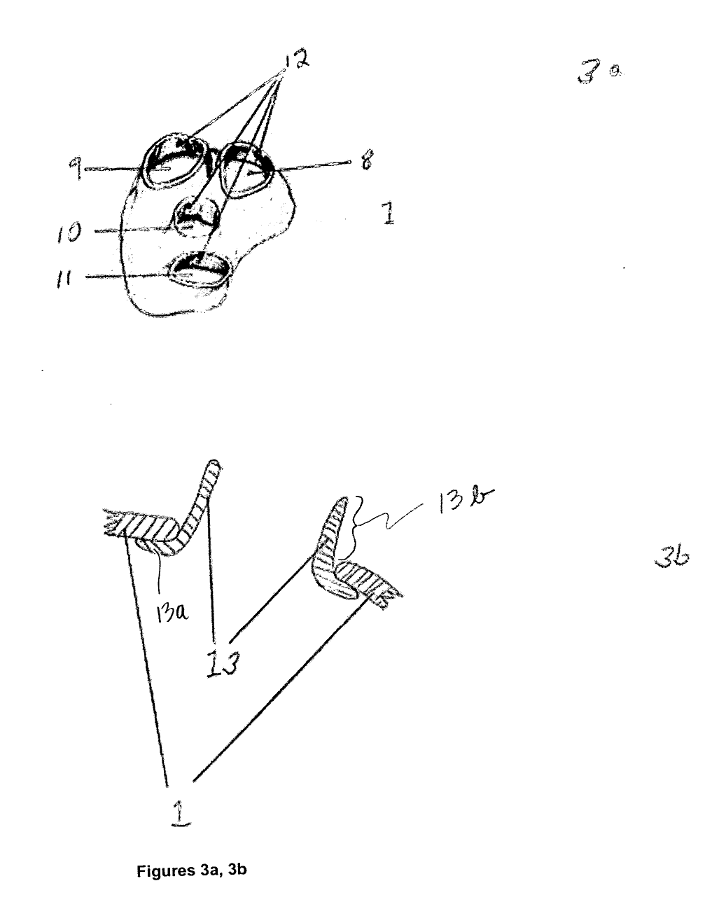

Referring now to FIGS. 3a-3b, an interface between dome 1 and respective atrial and great vessel walls is illustrated. In one embodiment, the interface between a heart and dome 1 may be accomplished by simple boundaries of the openings 8-11, which are shaped to fit the external walls of the atria 4,5 or great vessels 6, 7 at the base of the heart (see FIGS. 2a-2c). Alternative embodiments of the invention use openings having portions which are curved and extended for a variable distance distally on the walls of the vessels, as shown in FIGS. 3a-3b.

Referring to FIG. 3a, one embodiment of the dome 1 incorporates integral extensions 12 that extend from the boundaries of the opening 8-11. The flared extensions 12 may be used on all such openings, or may be used selectively on one or more of the openings, such as to surround the great vessels 6, 7. As such, the flared extensions may be formed integrally with the dome. Alternatively, sleeves 13 might be utilized, as illustrated in FIG. 3b.

The configuration with flared extensions 12 or sleeves 13 will distribute any compressive or tensile forces over larger areas than a simple boundary such as a cut surface. As illustrated in FIG. 3b, the embodiment using separate sleeves may use sleeves 13, which are placed about one or more of the atria and great vessels. The cross-section of sleeves may resemble a top-hat with the ‘brim’ 13a of the ‘hat’-positioned, and optionally secured, on the cardiac side of the respective dome 1 opening and an extension portion 13b along the outer surface. As noted with respect to the embodiment is FIG. 3a, the sleeves 13 may be used with all of the respective dome openings 8-11 or might selectively be used with one or more of those openings. The sleeve 13 of FIG. 3b is made as a separate piece from the dome 1, but is then appropriately secured with the dome, as shown to work in conjunction therewith.

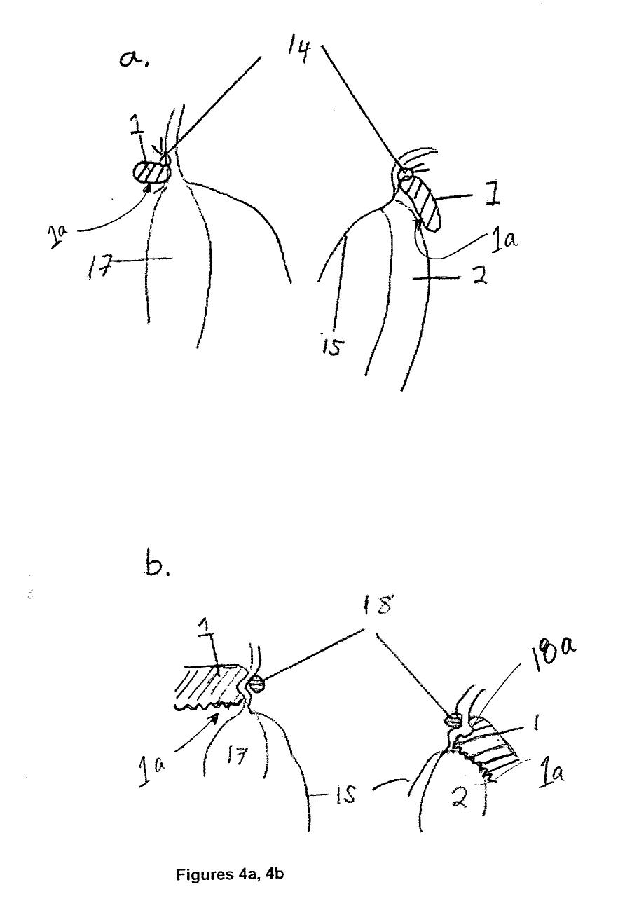

Fixation of the dome 1 to the outer walls of the atrial and/or great vessel walls of the heart (see FIGS. 1a, 1c and 1d) may be accomplished in a number of ways, as illustrated by FIGS. 4a-4b. For example, the dome 1 might be secured with simple sutures 14. In FIG. 4a, the sutures 14 are shown fastening an inner margin of dome 1 proximate to mitral valve 15. Such sutures might be put in place using appropriate cardiothoracic techniques. In another embodiment, securement may be accomplished with internal locking sutures. For example, semi-rigid rings 18 that interlock may be used. The rings 18 interlock through the atrial or great vessel wall. A mating shape, such as a groove or indent 18a, is formed on the internal margin of the dome opening(s) to receive ring 18 or any other interlocking structure (FIG. 4b). The locking structures are therefore located internally of the atria or great vessels. Securement might also be accomplished by any other stapling, gluing, pinning, ligating, suturing, or other technique known to those familiar with surgical techniques and devices. Furthermore, active securement might be eliminated, if experience demonstrates that is safe and effective to simply interface the dome with the heart atria and great vessels as illustrated in FIG. 1a.

In addition to any immediate means of mechanical fixation, the surfaces 1a of the dome contacting the appropriate atrium or great vessel may be textured or otherwise configured to facilitate and encourage biologic fixation through ingrowth of healing tissue, as shown in FIG. 4b.

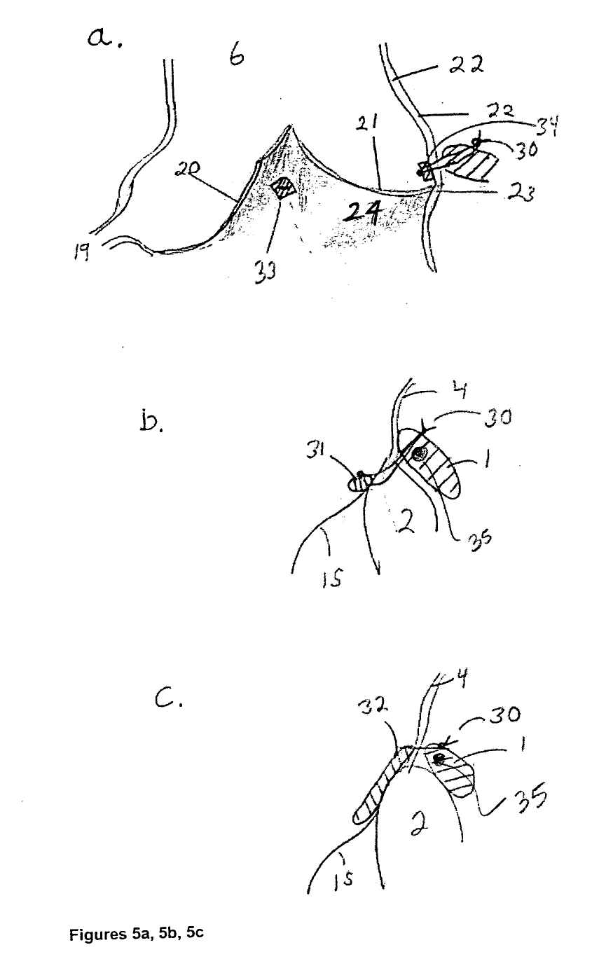

In another embodiment of the invention, the dome 1 may be fixed or mechanically stabilized with the cardiac fibrous skeleton of the heart. Referring to FIGS. 5a-5c, the dome 1 is shown stabilized relative to the fibrous skeleton of the heart. For example, in the illustrated embodiment, the valve annuli, which are part of that skeleton are used for securement. This may be done in several ways, which are non-limiting examples of securement. For example, simple or ‘mattress’ or other sutures between force-distributing members on the valve annuli and the inner margins of the openings 8-11 in the dome 1 may be utilized. These may include, but are not limited to various securement structures. For example, for the semilunar, or ventricular outflow valves (aortic and/or pulmonic), pledgets 34 are positioned adjacent the commissures 23 associated with the respective valve. Sutures anchored with the pledgets pass through the vessel wall, such as wall 22, to the margins of the respective dome opening (FIG. 5a).

Referring to FIG. 5a, the pledgets 34 may be used at various points around the valve annuli for securing the dome proximate their respective opening 8-11 in the dome. Pledgets 34 or other appropriate securement structures anchor the dome in the proper position on the heart, such as illustrated in FIG. 1a. As discussed above with respect to FIGS. 3a and 3b, dome 1 may incorporate one or more extensions 12 or sleeves 13, which are not shown in FIGS. 5a-5c. With the separate sleeve 13, the suture 30 may pass through the sleeve, as well. The pledgets 34 may be formed of a Teflon®, felt, or some other similar material that is biocompatible with the heart and its internal environment.

Alternatively, for the atrioventricular valves (mitral and/or tricuspid), standard or modified anuloplasty rings 31 might be used as a securement structure. Sutures 30 are sewn through or pierce the rings and the annular tissue of the valves. The sutures extend through the atrial wall to the margins of the respective dome opening as shown in FIG. 5b.

In another embodiment, semi-rigid, generally conical valve rings 32 shaped on one margin to adapt to the atrioventricular (mitral and/or tricuspid) annulus, and being sufficiently wide to extend to the plane of the dome margin might be used as shown in FIG. 3c. The outer margin (i.e., that away from the valve interface) may then be coupled or sewn through the atrial wall with interrupted or continuous sutures 30 to the inner margins of the dome opening(s), as shown in FIG. 5c.

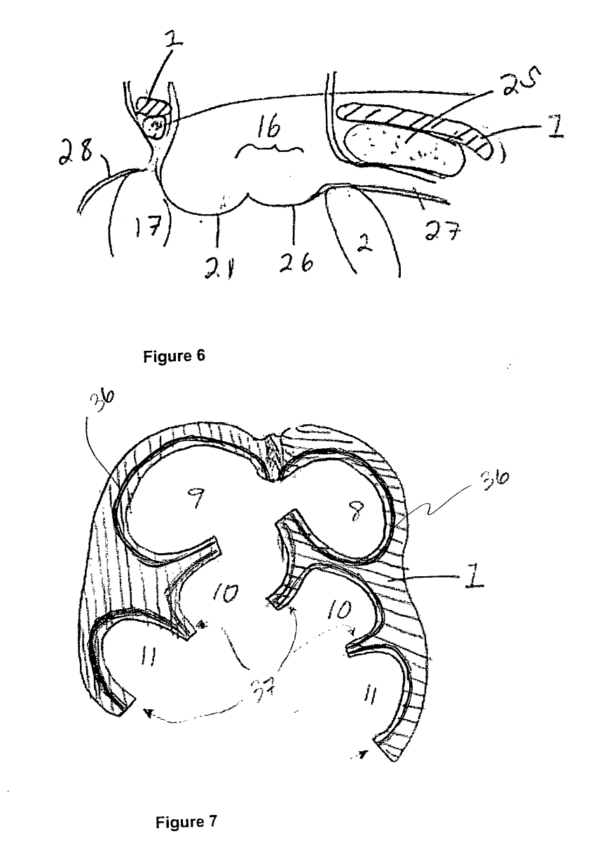

In accordance with another aspect of the invention, cushioning may be used to protect cardiac tissue at the base of the heart, particularly the proximal portions of the coronary arteries, cardiac veins, and coronary sinus, when the dome 1 is used. For example, cushion 25 is conformable and intended to conform to the topography of the basal margin of the ventricular mass with its overlying coronary vessels to minimize compressive effects of the dome 1 on them during installation and actuation of the overall device. Alternatively, rather than a separate cushion 25, the dome 1 may include, on at least part of the ventricle-facing surface, a compliant ‘pillow’ with a flexible skin, preferably of a fabric or a polymer membrane and a filling. Referring to FIG. 6, the cushion 25 may be integral with dome 1. Nonlimiting examples of the filling materials are a liquid, such as saline or a silicone oil, a gel, such as a silicone gel, or multiple approximately spherical or multi-faceted beads. In the latter case, the pillow or cushion may be described as a ‘beanbag’. In one embodiment, the cushion is coextensive with the entire dome and surround a single opening or all 2 or 4 openings. Alternatively, the cushion 25 may be ‘doughnut’ shaped and may surround individual great vessels (most likely the aorta, considering coronary artery origins) and/or atria, rather than covering the entire dome. Therefore, the cushion(s) 25 may be intrinsic or integral to the structure of the dome or separate from the dome.

Regarding reinforcements for openings in the dome 1, in the event that computations of forces or experimental evidence indicates that traction from an attached actuator mechanism, when actuated, will unfavorably distort the margins of an atrial or great-vessel opening in the dome, the dome near the opening(s) may be reinforced with a rigid, semi-rigid, or spring-like ring structure 35 (See FIGS. 5b, 5c, and 7). The ring structure may be positioned around one or more openings, although FIG. 7 shows ring structures 35 around all of the openings.

To allow controlled elastic deformation of the margins of the dome 1, these margins may be reinforced with an elastic member such as the corrugated metallic member or ‘zig-zag wire’ described in a patent application filed Jul. 18, 2002 entitled “A Flexible Torsionable, Cardiac Framework For Heart Wall Actuation of the Natural Heart.” which is incorporated by reference herein. The reinforcement may extend about all or part of the margin.

In another embodiment, dome 1 may have a construction for placement on a heart in situ—i.e., not explanted with intent to autotransplant. (See FIG. 7) The dome 1, and any associated accessory members, may be made so that they may be secured around still-intact atria and great vessels. This type of placement is accomplished by separation and reattachment of one margin of the opening(s) as shown in FIG. 7. That is, the dome is separated into portions or sections for positioning proximate the atria and/or great vessels. If any ring structures 35 are used with such a come, one margin of this ring structure(s) would need to have the equivalent of a hinge or local point 36 of flexibility, and the other side have a point 37 of separation and reattachment. These points of reattachment could be sutures, snaps, staples, clips, or any other fastening mechanism familiar to those acquainted with the arts and sciences of mechanical engineering design and/or any field of surgery.

The dome of the present invention will be utilized in conjunction with other elements 29 for actuation of the heart. These other elements may be or are, anchored to the dome. Such elements could be additional stabilizing elements or actuating elements.

For example, in accordance with one aspect of the present invention, an interventricular (vertical) yoke arc may be coupled to the dome 1. For example, the vertical portion of the yoke as shown in U.S. Pat. No. 5,957,977, may be used in conjunction with the dome to provide an external framework for the heart. In such a scenario, the dome would essentially replace the generally horizontal arc portion of the yoke. Alternatively, the actuator mechanism or mechanisms might also be attached to the dome 1, such as the mechanism illustrated in U.S. patent application Ser. No. 09/850,554, filed May 7, 2001, and entitled, “Heart Wall Actuation Device for the Natural Heart,” which application is incorporated herein by reference. Shape-limiting elements might also be coupled to the dome, as illustrated in U.S. patent application Ser. No. 10/223,271, filed Aug. 19, 2002, and entitled, “Heart Wall Actuation System for the Natural Heart with Shape-Limiting Elements, which application is incorporated herein by reference. Essentially, structures coupled to the yoke of U.S. Pat. No. 5,957,977, may also be coupled to basal dome 1.

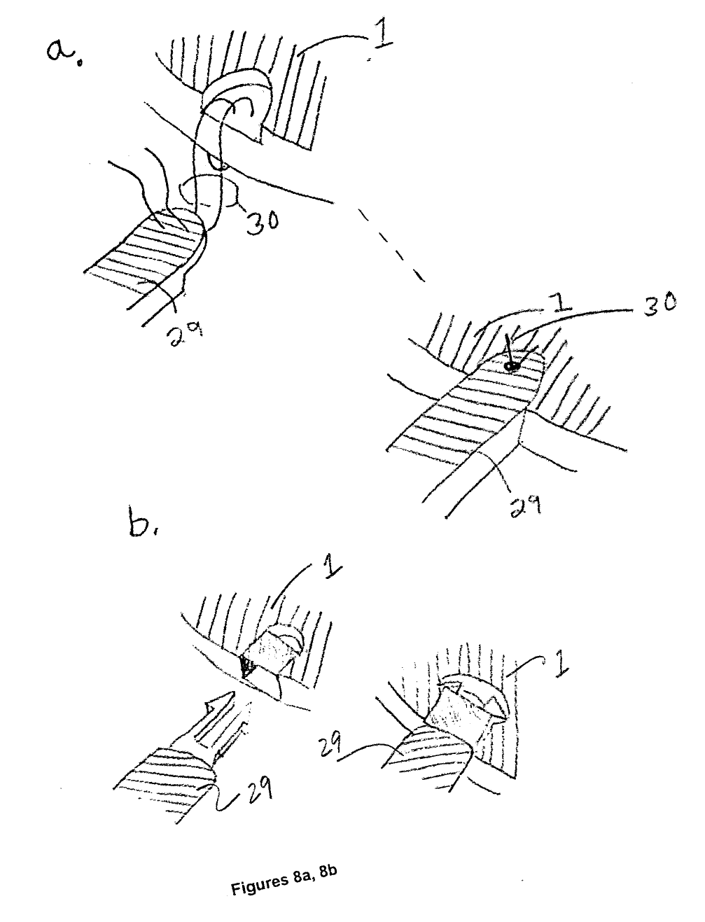

FIGS. 8a and 8b illustrate several examples of attachment structures or mechanisms for attaching other elements to dome 1. For example, sutures 30 might be used or some other kind of structure like a clip structure 38 may be used. Of course, as understood by a person ordinarily skilled in the art, other attachments could also be utilized. Furthermore, the structure 29 might be integrally formed with the dome 1. The dome provides an anchor for such elements.

Referring to FIG. 8a, the composition of at least the margins of the dome 1 include material configured to allow suturing, such as a fabric, or fiber-reinforced, or fabric-reinforced elastomer. The element 29 is appropriately sutured to dome 1. Slots or channels may be used which allow element 29 to ‘click-in’ the dome at pre-determined discrete points. Hook-and-loop fasteners, such as Velcro®, might also be used to couple the dome with other elements. Holes, with or without premounted screws, intended to be bolted with nuts and/or washers to the other components, might also be used. Other means of fixation, reversible or irreversible, such as will be know to those familiar with the arts and sciences of surgery and/or mechanical engineering can be used, as well.

What is claimed is:

1. An actuation system for assisting the operation of a natural heart comprising:
a dome structure configured for being coupled with a ventricular portion of the heart, the dome structure having at least one opening formed therein;
the dome structure, proximate the opening, being configured to interface with at least one of an atrial chamber and a great vessel of the heart; and
one of a stabilizing element and an actuating element being anchored to the dome structure for engaging a portion of the heart.

2. The actuation system of claim 1 wherein the dome structure is formed of a flexible material.

3. The actuation system of claim 1 wherein the dome structure is formed of a generally rigid material.

4. The actuation system of claim 1 further comprising a locking structure positioned internally of at least one of the atria or great vessels, the dome structure configured for interfacing with the locking structure through a wall of one of the atria or great vessels

5. The actuation system of claim 1 further comprising a plurality of openings formed in the dome structure.

6. The actuation system of claim 1 further comprising:
a suture structure positioned internally of at least one of the atria and great vessels; and
sutures spanning a wall of one of the atria and great vessels and anchoring the dome structure with the suture structure.

7. The actuation system of claim 6 wherein the suture structure is an angioplasty ring.

8. The actuation system of claim 1 further comprising a cushion positioned between the dome structure and the heart.

9. A heart-mounted structure for assisting the operation of a natural heart comprising:
a dome structure configured for being coupled a basal surface of a ventricular portion of the heart, the dome structure having at least one opening formed therein; and
the dome structure, proximate the opening, being configured to interface with at least one of an atrial chamber and a great vessel of the heart.

10. The heart-mounted structure of claim 9 wherein the dome structure is formed of a flexible material.

11. The heart-mounted structure of claim 9 wherein the dome structure is formed of a generally rigid material.

12. The heart-mounted structure of claim 9 wherein the dome structure is configured for interfacing, through a wall of one of the atria or great vessels, with a locking structure positioned internally of at least one of the atria or great vessels.

13. The heart-mounted structure of claim 9 further comprising a plurality of openings formed in the dome structure.

14. The heart-mounted structure of claim 9 wherein the dome structure includes an extension section extending therefrom to interface with at least one of the atria and great vessels.

15. The heart-mounted structure of claim 9 further comprising a sleeve for interfacing with at least one of the atria and great vessels, the dome structure holding the sleeve in place when positioned on the heart.

16. The heart-mounted structure of claim 9 wherein the dome structure includes a textured surface for interfacing with a heart surface.

17. The heart-mounted structure of claim 9 further comprising a cushion for positioning between the dome structure and the heart.

18. The heart-mounted structure of claim 17 wherein the cushion is coextensive with the entire dome.

19. The heart-mounted structure of claim 17 wherein the cushion interfaces with an opening in the dome.

20. The heart-mounted structure of claim 17 wherein the cushion is integral with the dome.

21. The heart-mounted structure of claim 9 further comprising a ring structure positioned around the opening in the dome structure.

22. The heart-mounted structure of claim 9 wherein the dome structure includes separated sections for positioning the dome structure proximate the atria or great vessels.

23. The heart-mounted structure of claim 9 further comprising at least one of a stabilizing element and an actuating element, the dome structure configured for interfacing with such an element to anchor the element.

24. The heart-mounted structure of claim 23 wherein the one of a stabilizing element and actuating element are integrally formed with the dome structure.
